(19) United States
(12) Patent Application Publication
(10) Pub. No.: US 2012/0156128 A1
(43) Pub. Date: Jun. 21, 2012

(54) CONTROLLABLE RELEASE COMPOSITION AND METHOD FOR PREPARING SAME

(76) Inventors: Yu-Li CHANG, Hsinchu City (TW); Yuan-Chia Chang, Hsinchu City (TW); Min-Ying Lin, Hsinchu City (TW); Xi-Zhang Lin, Tainan City (TW)

(21) Appl. No.: 13/316,050

(22) Filed: Dec. 9, 2011

Related U.S. Application Data
(60) Provisional application No. 61/422,126, filed on Dec. 10, 2010.

(30) Foreign Application Priority Data
Dec. 2, 2011 (TW) 100144309

(51) Int. Cl. A61K 51/06; A61K 31/704; A61K 31/337; A61P 35/00

(52) U.S. Cl. 424/1.11; 514/34; 514/449

(57) ABSTRACT

A controllable release composition is provided, including a polymer substrate and an active ingredient, in which the polymer substrate is a polymer blend including a biodegradable polyester, polyanhydride, and/or polyether, and the active ingredient includes a radioactive agent or a chemotherapeutic agent. The controllable release composition is useful for cancer therapy, particularly for solid cancer treatment.

### CROSS REFERENCE TO RELATED APPLICATIONS

This application claims the benefit of U.S. Provisional Application No. 61/422,126, filed Dec. 10, 2010, and Taiwan Patent Application No. 100144309, filed Dec. 2, 2011.

The disclosure of the application is incorporated herein by reference.

### BACKGROUND OF THE INVENTION

1. Field of the Invention

The present invention relates to a controllable release composition composed of a polymer substrate and an active ingredient, and in particular relates to a use of the controllable release composition on cancer therapy.

2. Description of the Related Art

Effective drug treatment refers to treatment wherein active components of a drug are present in an effective amount at a target organ after administration. However, most drugs may be acidically or enzymatically degraded when passing through gastrointestinal tracts or may be unable to pass through the blood-brain barrier. There is also a problem where several drugs lose activities when reaching a target organ. Thus, therapeutic results may be unsuccessful and continued administration would be required in order to maintain the effective amount of the active component of the drug at the target organ.

Generally, drugs in a well-soluble solvent are easily disintegrated, leading to an initial burst release of the active component of the drug. Thus, such drugs, are incapable of continually releasing the active component in a specific amount during a period of time. Current research aims to encapsulate drugs with polymers to adjust the release profile of the active component of the drug. However, if the polymers cannot be disintegrated or degraded in the body, the active component of the drug may not be released and therefore cause a burden to the body or result in side effects. Thus, the optimal relationship between polymer disintegration and drug release is one of the research and development areas which are being studied.

Radioembolization (RE), also named selective internal radiation therapy (SIRT), is widely-used in the treatment of liver cancer in clinics. The effect of SIRT may depend on the selection of patients. The treatment needs preventive strategies like a detailed test with angiography in advance and blocks the hepatic artery branch supplying blood to gastrointestinal tracts in order to avoid radiation damage on the gastrointestinal tracts. For instance, angiography with technetium-labeled macroaggregated albumin (^{99m}Tc-MAA) is required before SIRT is performed to estimate the possible flow of the radioactive agent from the lungs during treatment. When the radioactive flow from the lungs is greater than 20%, it is not preferable to perform the yttrium-90 radioembolization. Hence, more care is needed to prevent radiation damage on normal cells, tissues or organs when carrying on with SIRT. The goal is to hold the radioactive agent at the cancerous tissue and possibly decrease the release of the radioactive agent.

Taylor, R. R. et al. described the treatment of liver cancer with a drug eluting bead (DEB) loaded with irinotecan, which showed the same effect of the yttrium-90 radioembolization (European Journal of Pharmaceutical Sciences 30, 7-14, 2007). An in vitro release profile was reported showing that the DEB-loaded irinotecan was completely released in a half hour to 4 hours. This showed that irinotecan loaded in DEB needed to be totally released or released to the effective concentration in a short time in order to achieve the therapeutic effect.

U.S. Pat. No. 5,919,835 provides a formulation of a polymer mixture as a carrier carrying therapeutically active component. The polymer mixture consists of polyesters, polyanhydrides or combinations thereof with active components, such as antibiotics, anti-inflammatories, anesthetics, antivirals, antifungals, antihypertensives, antiarrhythmics and neuro-active agents, to control different release rates (Abstract and claims 1-12).

U.S. Pat. No. 7,160,551 B2 provides an injectable composition for avoiding burst release of a bioactive agent. The injectable composition contains a bioactive agent, an non-aqueous biocompatible solvent, a hydrophobic polymer and an amphiphilic block copolymer comprising at least one segment of polyethylene oxide) and at least one segment of poly(propylene oxide) (Abstract and claim 1).

U.S. Pat. No. 7,964,219 B2 discloses a composition comprising a complex of a biologically active compound having at least one basic functional group and a polyanion derived from hexahydroxyclohexane having at least two negatively charged functional groups. By complexing a biologically active compound with a polyanion, the tight, stable complex may be incorporated into a long-acting dosage system having a more desired drug release curve over time (Abstract).

Based on the prior arts, the inventors have further focused on the controlled release formulation of bioactive ingredients for cancer therapy. Particularly, the present invention aims to achieve the standards required by clinics for disintegration rates and release rates of a bioactive agent by mixing the bioactive agent well-used in radiation therapy and chemotherapy with a specific polymer blend to increase therapeutic effects and become more convenient for clinical use.

### BRIEF SUMMARY OF THE INVENTION

The invention provides a controllable release composition, comprising a mixture of a polymer substrate and a radioactive agent, wherein the polymer substrate is a polymer blend comprising a biodegradable polyester and polyanhydride.

The invention further provides a controllable release composition, comprising a mixture of a polymer substrate and a chemotherapeutic agent, wherein the polymer substrate is a polymer blend comprising a biodegradable polyester, polyanhydride and polyether.

The invention further provides a method for preparing the controllable release composition above, comprising dissolving or dispersing the polymer blend in a first solvent to form a first solution or suspension, dissolving or dispersing the active ingredient (radioactive agent or chemotherapeutic agent) in a second solvent to form a second solution or suspension, mixing the aforementioned first and second solutions or suspensions to form a homogeneous or uniformly dispersed mixture, heating the mixture to vaporize the first and second solvent, and therefore obtaining the controllable release composition.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows the in vitro cumulative release amounts of an active ingredient (NaReO₄) over time from the composition, for five compositions containing five different polymer substrate formulations, respectively, in one example of the present invention;

FIG. 2 shows the in vitro cumulative release amounts of an active ingredient (Re—Sn colloid microparticles) over time from the composition, for ten compositions containing ten different polymer substrate formulations, respectively, in one example of the present invention;

FIG. 3 shows the in vitro cumulative release amounts of an active ingredient (doxorubicin) over time from the composition, for four compositions containing four different polymer substrate formulations, respectively, in one example of the present invention;

FIG. 4 shows the in vitro cumulative release amounts of an active ingredient (paclitaxel) over time from the composition, for four compositions containing four different polymer substrate formulations, respectively, in one example of the present invention

FIG. 5 shows the in vitro cumulative release amounts of an active ingredient (Y₂O₃) over time from the composition, for four compositions containing four different polymer substrate formulations, respectively, in one example of the present invention;

FIG. 6 shows the in vitro cumulative release amounts of an active ingredient (YCl₃) over time from the composition, for four compositions containing four different polymer substrate formulations, respectively, in one example of the present invention; and

FIG. 7 shows the in vitro cumulative weight losses of the composition over time, for eight compositions containing eight different polymer substrate formulations, respectively, in one example of the present invention.

### DETAILED DESCRIPTION OF THE INVENTION

The following description is of the best-contemplated mode of carrying out the present invention. This description is made for the purpose of illustrating the general principles of the invention and should not be taken in a limiting sense. The scope of the present invention is best determined by reference to the appended claims.

The controllable release composition according to the present invention shows a controlled disintegration rate and cumulative disintegration amount of the polymer substrate and a controlled release rate and cumulative release amount of the active ingredient, which are achieved by mixing a blend of a biodegradable polyester, a biodegradable polyanhydride, and/or a biodegradable polyether with an active ingredient exhibiting therapeutic effects.

The polyesters, polyanhydrides and polyethers according to the present invention include but not specifically limited to all biocompatible and biodegradable polyesters, polyanhydrides, and polyethers. However, the biomaterials which have been approved for practice in humans or animals are preferable ones in the present invention.

Specifically, the polyesters may comprise polycaprolactone (PCL), polyvalerolactone (PVL), polypropiolactone (PPL), polybutyrolactone (PBL), poly(lactide-co-glycolide (PLGA), polylactic acid (PLA), polyglycolide (PGA), poly(isobutylcyanoacrylate (PIBCA), polyisophthalic acid (PIPA), poly-1,4-phenylene dipropionic acid (PPDA), poly(mandelic acid) (PMDA), poly(propylene fumarate (PPF), poly(ortho ester) (POE), or combinations thereof.

The polyanhydrides may comprise poly(sebacic anhydride) (PSA), poly-(bis-(p-carboxyphenoxy)propane anhydride (PCPPA), poly-(bis(p-carboxy)methane anhydride) (PCMA), poly-carboxyphenoxypropane-co-sebasic acid (p(CPP-SA)), poly-carboxyphenoxypropane-co-isophthalic acid (p(CPP-IPA)), poly(fatty acid dimmer-co-sebacic acid (p(FAD-SA)), or combinations thereof.

The polyethers may comprise poly(ethylene glycol) (PEG), poly(propylene glycol) (PPG), poly(butylene-s glycol) (PBG), or combinations thereof.

When the polymer blend comprises a polyester and a polyanhydride, the polymer blend preferably comprises about 50 parts by weight of the polyester and about 5˜70 parts by weight of the polyanhydride, but is not limited thereto. The polymer blend may further comprise a polyether and the polymer blend preferably comprises about 50 parts by weight of the polyester, about 5˜70 parts by weight of the polyanhydride, and about 5˜70 parts by weight of the polyether. In one example, the polymer blend comprises about 50 parts by weight of the polyester, about 30˜40 parts by weight of the polyanhydride, and about 30˜40 parts by weight of the polyether. However, the ratio among the parts of weight of the polyester, those of the polyanhydride, and those of the polyether may be slightly modified, depending on what kind of the active ingredient release profile is to be obtained.

When the polymer blend comprises much more of the polyester portion, the polymer blend may not be easily disintegrated, because the polyester degradation rate is usually relatively low. While, if the polymer blend comprises less of the polyester portion, the polymer blend may be quickly disintegrated and the active ingredient quickly released within a short period of time. Because the polyesters usually exhibit poor degradation rates, by blending the polyanhydrides and/or polyethers with the polyesters, the disintegration (in terms of the disintegration rate and the cumulative disintegration amount) of the polymer blend can be modulated and therefore the release of the active ingredient (in terms of the release rate and the cumulative release amount) controlled.

The controllable release composition according to the invention may be prepared by a method comprising the following steps:
(1) dissolving or dispersing the polymer blend with a first solvent to form a first solution or suspension;
(2) dissolving or dispersing the active ingredient with a second solvent to form a second solution or suspension;
(3) mixing the first solution or suspension and the second solution or suspension to form a mixture;
(4) heating the mixture to vaporize the first and second solvent; and
(5) obtaining the controllable release composition.

The term “dissolve” refers to that each of the constituent polymers forming the polymer blend or the active ingredient is well solved in a solvent to form a homogeneous solution. The term “disperse” refers to that each of the constituent polymers forming the polymer blend or the active ingredient is poorly solved in a solvent but instead can be suspended in the solvent to form a uniformly dispersed suspension. It is not limited to dissolve or disperse the constituent polymers forming the polymer blend or the active ingredient according to the present invention. All the methods involved in dissolution and dispersion, which can make the solutions or suspensions of each of the constituent polymers forming the polymer blend and that of the active ingredient form a homogeneous solution or a uniformly dispersed suspension, when mixed, are included in the present invention.

The “first solvent” refers to a solvent in which each of the constituent polymers forming the polymer blend can be homogeneously dissolved or uniformly dispersed. The first kind solvent can be exemplified as acetone, acetonitrile, chloroform, dichloromethane, ethyl acetate, isopropanol, methanol, tetrahydrofuran, or combinations thereof. The concentration of each of the constituent polymers forming the polymer blend in the solution or suspension made of it and its first solvent is not specifically limited. In one example, the concentration of each of the constituent polymers forming the polymer blend in the solution or suspension made of it and its first solvent is in the range of 50˜300 mg/ml.

The “second solvent” refers to the solvent in which the active ingredient can be homogeneously dissolved or uniformly dispersed. The second kind solvent can be exemplified as water, acetone, acetonitrile, chloroform, dichloromethane, ethyl acetate, isopropanol, methanol, ethanol, tetrahydrofuran, or combinations thereof. The concentration of the active ingredient in the solution or suspension made of it and its second kind solvent is not specifically limited. In one example, the concentration of the active ingredient in the solution or suspension made of it and its second kind solvent is in the range of 1˜100 mg/ml. In addition, the preferable ratio between the weight of the polymer substrate and that of the active ingredient is but not limited to in the range of 100:0.0002˜100:22.

After the solutions or suspensions made of the constituent polymers forming the polymer blend and their respective first solvents and that made of the active ingredient and its second solvent are homogeneously mixed or uniformly dispersed, the solvent(s) are vaporized during the heating step, then the controllable release composition according to the invention can be obtained. The temperature of the heating step can be appropriately selected or adjusted without destroying the physical and chemical properties of the polymer substrate and those of the active ingredient. The preferable temperature is in the range of 40˜80° C. for the heating step, but is not limited thereto.

The “active ingredient” refers to a substance with therapeutic effects. There is no specific limitation to the active ingredient, but preferably, it is a substance with activity on cancer therapies. It is well known that radioactive materials are able to treat cancers by teletherapy or brachytherapy. Teletherapy, also named external beam radiotherapy, kills tumor cells by widely irradiating the tumor or cancerous tissue through the skin surface of humans or animals, which is commonly used in clinics. However, teletherapy destroys systematic normal cells at the same time it kills tumor cells. Brachytherapy refers to a therapy where the radioactive agent is placed close to or implanted into the tumor or cancerous tissue. According to brachytherapy, the radioactive agent emits higher radiation on the tumor or cancerous tissue to kill the cancerous cells, but there is lower radiation on the surrounding tissues so that the damage to the normal cells is decreased. According to brachytherapy, the radiotherapy is not only precisely on the tissue requiring treatment but also decreases the damage to normal cells.

According to the present invention, the controllable release composition comprising a radioactive agent is suitable to be formulated into an implant dosage form for brachytherapy. Because it can exhibit a controlled disintegration rate and a controlled release rate, the controllable release composition of the present invention is able to keep the cumulative release amount of an effective radioactive agent under a safety threshold level allowed for use in human for a specific duration and maintain this state for a period of time, when implanted into the tumor or cancerous tissue. In addition, the controllable release composition comprising a chemotherapeutic agent can also be formulated into an implant dosage form to be implanted into tumors or tumor-surrounding tissues. As it can exhibit a controlled disintegration rate and a controlled release rate, the controllable release composition of the present invention is able to bring the cumulative release amount of an effective chemotherapeutic substance to the amount required for efficacious treatment within a short period of time, when implanted into the tumor or cancerous tissue.

The radioactive agent involved in the radioactive agent useful in clinical radiotherapy according to the present invention is exemplified like a radioactive seed of Co-60, Sr-89, I-125, Cs-137, Ir-192, Y-90, Re-188 or Ra-226, but is not limited thereto. Accordingly, the controllable release composition with a radioactive agent payload of the present invention can be useful for cancer therapy, and particularly, for solid tumor therapy.

The chemotherapeutic substance involved in the chemotherapeutic agent useful in clinical chemotherapy according to the present invention is exemplified like 5-fluorouracil, irinotecan, topotecan, bortezomib, lapatinib, trastuzumab, gemcitabine, methotrexate, doxorubicin, oxaliplatin, paclitaxel, camptothecin, cisplatin, bevacizumab, or combinations thereof, but is not limited thereto. Accordingly, the controllable release composition with a chemotherapeutic agent payload of the present invention can be useful for cancer therapy, and particularly, for solid tumor therapy.

The controllable release composition of the present invention may further comprise pharmaceutically acceptable carriers or excipients which are inert to the polymer substrate and the active ingredient, such as starch, sucrose, lactose, carboxymethyl cellulose, microcrystalline cellulose, mannitol, sorbitol, silica colloide, magnesium stereate, or the like. The pharmaceutically acceptable carriers or excipients can be added into the composition with an appropriate ratio according to commonly pharmaceutical practice.

### EXAMPLES

The compounds with abbreviations used in the following examples are listed below.

PCL: polycaprolactone

PSA: poly(sebacic anhydride)

PLGA: poly(lactide-co-glycolide)

PEG: poly(ethylene glycol)

NaReO₄: sodium perrhenate

DCM: dimethylchloride

Y₂O₃: yttrium oxide

YCl₃: yttrium(III) chloride

### Example 1

### Preparation of the Composition Encapsulating NaReO₄

PLGA, PCL, and PEG were respectively dissolved with the first solvents as listed in Table 1 to form polymer solutions with a concentration of 100 mg/ml. NaReO₄ was dissolved with the second solvents as listed in Table 1 to form NaReO₄ solutions with a concentration of 10 mg/ml.

Mixed together the above-mentioned constituent polymer solutions according to the formulations (five formulations) as listed in Table 1 to form the polymer substrate solutions. For each formulation, 1 ml of the polymer blend solution was uniformly mixed with 0.1 ml of the above-mentioned NaReO₄ solution. The mixture was then heated under 70° C. After the solvents were vaporized and the solid mixture was cooled, Samples 1-1˜1-5 as listed in Table 1 were obtained.

TABLE 1
The formulation of the polymer substrate used to encapsulate NaReO₄

| | | | | Solvents | | | | PCL/PLGA/PEG |
|---|---|---|---|---|---|---|---|---|
| Sample | PCL | PLGA | PEG | NaReO₄ | PCL | PLGA | PEG | (by weight) |
| 1-1 | 1 ml | — | — | water | DCM | — | — | 1/0/0 |
| 1-2 | 1 ml | — | — | acetone | acetone | — | — | 1/0/0 |
| 1-3 | 0.5 ml | 0.5 ml | — | acetone | acetone | DCM | — | 1/1/0 |
| 1-4 | 0.5 ml | — | 0.5 ml | acetone | isopropanol | — | isopropanol | 1/0/1 |
| 1-5 | 1 ml | — | — | — | DCM | — | — | 1/0/0 |

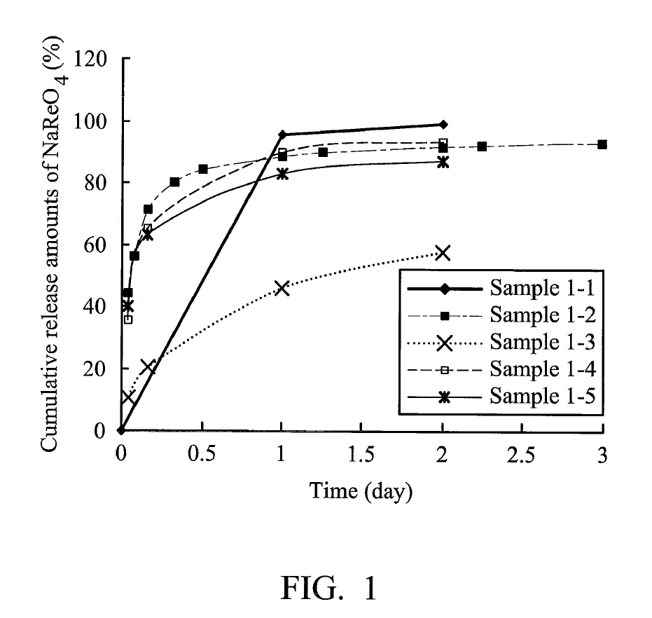

Subsequently, in vitro release tests were conducted. 10 mg of each of the above-mentioned samples was put into a vial filled with 20 ml of phosphate buffer. At the time point for analyses, 20 ml of the phosphate buffer in the vial was pipetted from the vial and the vial was replenished with another 20 ml of fresh phosphate buffer. The pipetted sample solutions were tested with ICP-AES to analyze the concentrations of rhenium (Re) within them. The cumulative release amount of the active ingredient was expressed in terms of percentage, which was calculated as follows: the amount of rhenium (Re) in the buffer solution divided by that in the sample and multiplied by 100. The results are shown in FIG. 1.

As shown in FIG. 1, the composition comprising only PCL as the polymer substrate (Samples 1-1, 1-2, and 1-5) rapidly released the active ingredient NaReO₄ within one day. The reason for such a fast release is perhaps because NaReO₄ is hydrophilic and well dissolved in water. The composition comprising a blend of PCL and PEG as the polymer substrate (Sample 1-4) also rapidly released the active ingredient NaReO₄ within one day. The reason for such a fast release is perhaps because both PEG and NaReO₄ are hydrophilic and well dissolved in water. The composition comprising a blend of PCL and PLGA as the polymer substrate (Sample 1-3) showed a release profile of the active ingredient NaReO₄ with an initial burst release at the beginning, followed by a slow release with a release rate decreasing over time. According to FIG. 1, it is noted that different polymer substrate formulations can result in distinct change of the initial burst release rates, the release rates thereafter, and the according cumulative release amounts. For radiotherapeutic agents, the cumulative release amount of the radioterapeutic substance for a specific period of time must be kept under a safety threshold level to prevent from damaging cells or tissues. According to the present invention, by modulating the formulation of the polymer substrate, the release amount of the radioactive agent can be controlled and kept under a safety threshold level and exhibit therapeutic effects.

### Example 2

### Preparation of the Composition Encapsulating Re—Sn Colloidal Microparticles

Preparation of Re—Sn Colloids

0.03 g of NaReO₄ and 0.3 g of SnCl₂ were respectively dissolved with 7.5 ml of a 0.1N HCl. Subsequently, the NaReO₄ solution and the SnCl₂ solution were mixed and placed in a 100° C. water bath for 1 hour. At the time point for ending the reaction, 15 ml of 0.2M phosphate buffer was added for neutralization to end the colloid formation reaction. The suspension resulted from the aforementioned reaction was centrifuged at 3600 rpm for 10 minutes. The supernatant was removed and the Re—Sn colloidal microparticles were obtained from the precipitation.

Preparation of the Composition Encapsulating Re—Sn Colloidal Microparticles

PLGA, PCL, and PEG were respectively dissolved with the first solvents as listed in Table 2 to form polymer solutions with a concentration of 100 mg/ml. The Re—Sn colloidal microparticles obtained beforehand were dispersed with the second solvent as listed in Table 2 to form a Re—Sn colloidal microparticle suspension with a concentration of 10 mg/ml. Mixed together the above-mentioned constituent polymer solutions according to the formulations (ten formulations) as listed in Table 2 to form the polymer substrate solutions. For each formulation, 1 ml of the polymer blend solution was uniformly mixed with 0.2 ml of the above-mentioned Re—Sn colloidal microparticles suspension. The mixture was then heated under 70° C. After the solvents were vaporized and the solid mixture was cooled, Samples 2-1˜2-10 as listed in Table 2 were obtained.

TABLE 2
The formulation of the polymer substrate used to encapsulate Re—Sn colloidal microparticles

| | | | | Solvents | | | | PCL/PLGA/PEG |
|---|---|---|---|---|---|---|---|---|
| Sample | PCL | PLGA | PEG | Re—Sn | PCL | PLGA | PEG | (by weight) |
| 2-1 | 0.5 ml | 0.5 ml | — | acetonitrile | acetonitrile | chloroform | — | 5/5/0 |
| 2-2 | 0.5 ml | — | 0.5 ml | acetonitrile | acetonitrile | — | acetonitrile | 5/0/5 |
| 2-3 | 0.4 ml | 0.6 ml | — | acetonitrile | acetonitrile | chloroform | — | 4/6/0 |
| 2-4 | 0.4 ml | 0.5 ml | 0.1 ml | acetonitrile | acetonitrile | chloroform | acetonitrile | 4/5/1 |
| 2-5 | 0.4 ml | 0.4 ml | 0.2 ml | acetonitrile | acetonitrile | chloroform | acetonitrile | 4/4/2 |
| 2-6 | 0.4 ml | 0.3 ml | 0.3 ml | acetonitrile | acetonitrile | chloroform | acetonitrile | 4/3/3 |
| 2-7 | 0.4 ml | 0.2 ml | 0.4 ml | acetonitrile | acetonitrile | chloroform | acetonitrile | 4/2/4 |
| 2-8 | 0.4 ml | 0.1 ml | 0.5 ml | acetonitrile | acetonitrile | chloroform | acetonitrile | 4/1/5 |
| 2-9 | 0.4 ml | — | 0.6 ml | acetonitrile | acetonitrile | — | acetonitrile | 4/0/6 |
| 2-10 | 0.3 ml | 0.4 ml | 0.3 ml | acetonitrile | acetonitrile | chloroform | acetonitrile | 3/4/3 |

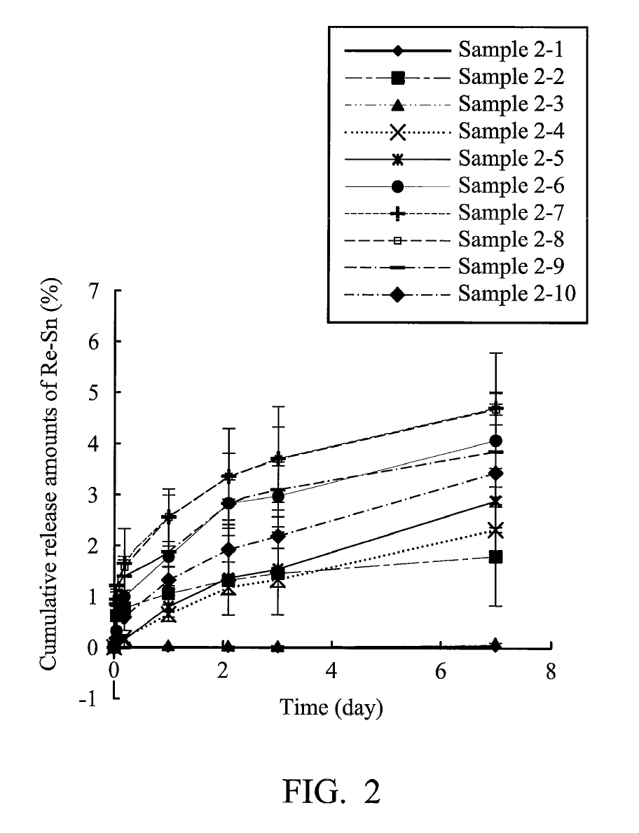

Subsequently, in vitro release tests were conducted. 10 mg of each of the above-mentioned samples was put into a vial filled with 20 ml of phosphate buffer. At the time point for analyses, 20 ml of the phosphate buffer in the vial was pipetted from the vial and the vial was replenished with another 20 ml of fresh phosphate buffer. The pipetted sample solutions were tested with ICP-AES to analyze the concentrations of rhenium (Re) within them. The cumulative release amount of the active ingredient was expressed in terms of percentage, which was calculated as follows: the amount of rhenium (Re) in the buffer solution divided by that in the sample and multiplied by 100. The results are shown in FIG. 2.

As shown in FIG. 2, the composition without PEG (Samples 2-1 and 2-3) released hardly any encapsulated active ingredient, i.e., Re—Sn colloidal microparticles. On the contrary, the composition comprising a blend of PCL and PEG as the polymer substrate (Samples 2-2 and 2-9) and that comprising a blend of PCL, PSA, and PEG as the polymer substrate (Samples 2-4˜2-8 and 2-10) showed a release profile of the active ingredient (Re—Sn colloidal microparticles) with an initial burst release at the beginning, followed by a slow release with a release rate decreasing over time. According to FIG. 2, it is noted that different polymer substrate formulations can result in distinct change of the initial burst release rates, the release rates thereafter, and the according cumulative release amounts. For radiotherapeutic agents, the cumulative release amount of the radiotherapeutic substance for a specific period of time must be kept under a safety threshold level to prevent from damaging cells or tissues. According to the present invention, by modulating the formulation of the polymer substrate, the release amount of the radioactive agent can be controlled and kept under a safety threshold level and exhibit therapeutic effects.

According to FIG. 2, the release profiles showed a trend that, when the polymer substrate was a blend of only PCL and PEG, the initial burst release rate and the according cumulative release amount of the active ingredient increased as the ratio of the PEG portion in the polymer substrate increased (e.g. Sample 2-2<Sample 2-9). When the polymer substrate was a blend of PCL, PSA, and PEG, the initial burst release rate and the according cumulative release amount of the active ingredient increased as the ratio of the PSA portion in the polymer substrate decreased (i.e. the ratio of the portion of PEG in the polymer substrate increased) (e.g. Sample 2-4<Sample 2-5<Sample 2-10<Sample 2-6<Sample 2-7=Sample 2-8).

### Example 3

### Preparation of the Composition Encapsulating Doxorubicin

PLGA, PCL, and PEG were respectively dissolved with the first solvents as listed in Table 3 to form polymer solutions with a concentration of 100 mg/ml. Doxorubicin was dissolved with the second kind solvent as listed in Table 3 (a mixture of water and ethanol at a ratio of 1:3 by volume) to form a doxorubicin solution of with a concentration of 50 mg/ml. Mixed together the above-mentioned constituent polymer solutions according to the formulations (ten formulations) as listed in Table 3 to form the polymer substrate solutions. For each formulation, 1 ml of the polymer blend solution was uniformly mixed with 0.1 ml of the above-mentioned doxorubicin solution. The mixture was then heated under 70° C. After the solvents were vaporized and the solid mixture was cooled, Samples 3-1˜3-4 as listed in Table 3 were obtained.

TABLE 3
The formulation of the polymer substrate used to encapsulate doxorubicin

| | | | | Solvents | | | | PCL/PLGA/PEG |
|---|---|---|---|---|---|---|---|---|
| Sample | PCL | PLGA | PEG | doxorubicin | PCL | PLGA | PEG | (by weight) |
| 3-1 | 1.0 ml | — | — | *ethanol aq. | acetonitrile | — | — | 1/0/0 |
| 3-2 | 0.4 ml | — | 0.6 ml | *ethanol aq. | acetonitrile | — | acetonitrile | 4/0/6 |
| 3-3 | 0.4 ml | 0.3 ml | 0.3 ml | *ethanol aq. | acetonitrile | chloroform | acetonitrile | 4/3/3 |
| 3-4 | 0.4 ml | 0.6 ml | — | *ethanol aq. | acetonitrile | chloroform | — | 4/6/0 |

*refers to ethanol aqueous solution.

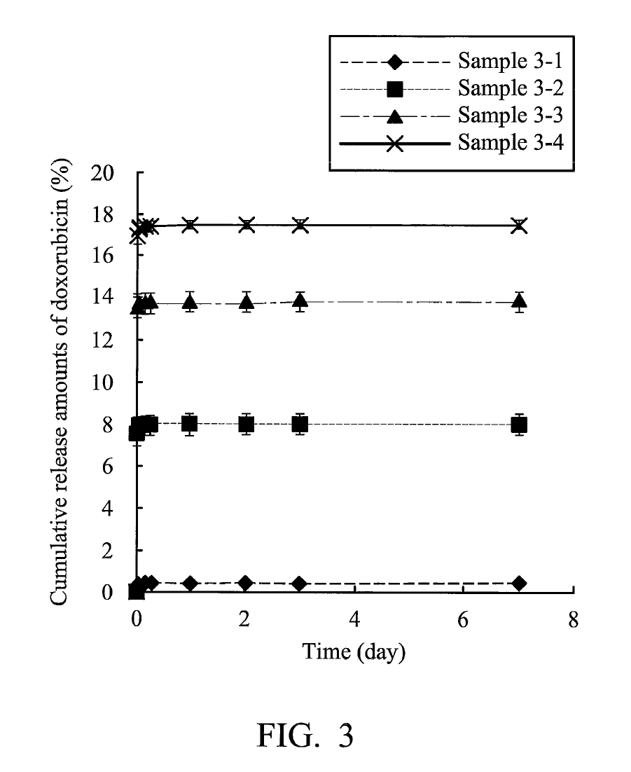

Subsequently, in vitro release tests were conducted. 10 mg of each of the above-mentioned samples was put into a vial filled with 20 ml of phosphate buffer. At the time point for analyses, 20 ml of the phosphate buffer in the vial was pipetted from the vial and the vial was replenished with another 20 ml of fresh phosphate buffer. The pipetted sample solutions were tested with HPLC to analyze the concentrations of doxorubicin within them. The cumulative release amount of the active ingredient was expressed in terms of percentage, which was calculated as follows: the amount of doxorubicin in the buffer solution divided by that in the sample and multiplied by 100. The results are shown in FIG. 3.

As shown in FIG. 3, the composition comprising only PCL as the polymer substrate (Sample 3-1) released few encapsulated active ingredient, i.e., doxorubicin. On the contrary, the composition comprising a blend of PCL and PSA as the polymer substrate, that comprising a blend of PCL and PEG as the polymer substrate, and that comprising a blend of PCL, PSA, and PEG as the polymer substrate (Samples 3-4, 3-2, and 3-3, respectively) showed release profiles of the active ingredient (doxorubicin) with different initial burst rates at the beginning and with slow release rates which were almost zero after the initial burst release. According to FIG. 3, it is noted that different polymer substrate formulations can result in distinct change of the initial burst release rates, the release rates thereafter, and the according cumulative release amounts. For chemotherapeutic agents, the cumulative release amount of the chemotherapeutic substance must be brought to a certain level required for efficacious treatment within a short period of time, when implanted into the tumor or cancerous tissue. According to the present invention, by adjusting the polymer substrate formulation, the release amount of the chemotherapeutic substance can be controlled and brought to a level required for exhibiting therapeutic effect, within a short time period.

According to FIG. 3, the release profiles showed a trend that the initial burst release rate and the according cumulative release amount of doxorubicin decreased as the ratio of the PSA portion in the polymer substrate decreased (e.g. Sample 3-1<Sample 3-2<Sample 3-3<Sample 3-4).

### Example 4

### Preparation of the Composition Encapsulating Paclitaxel

PLGA, PCL, and PEG were respectively dissolved with the first solvents as listed in Table 4 to form polymer solutions with a concentration of 100 mg/ml. Paclitaxel was dissolve with the second kind solvent as listed in Table 4 (acetonitrile) to form a paclitaxel solution with a concentration of 200 mg/ml. Mixed together the above-mentioned constituent polymer solutions according to the formulations (four formulations) as listed in Table 4 to form the polymer substrate solutions. For each formulation, 1 ml of the polymer blend solution was uniformly mixed with 0.1 ml the above-mentioned paclitaxel solution. The mixture was then heated under 70° C. After the solvents were vaporized and the solid mixture was cooled, Samples 4-1˜4-4 as listed in Table 4 were obtained.

TABLE 4
The formulation of the polymer substrate used to encapsulate paclitaxel

| | | | | Solvents | | | | PCL/PLGA/PEG |
|---|---|---|---|---|---|---|---|---|
| Sample | PCL | PLGA | PEG | paclitaxel | PCL | PLGA | PEG | (by weight) |
| 4-1 | 1.0 ml | — | — | acetonitrile | acetonitrile | — | — | 1/0/0 |
| 4-2 | 0.4 ml | 0.6 ml | — | acetonitrile | acetonitrile | chloroform | — | 4/6/0 |
| 4-3 | 0.4 ml | 0.3 ml | 0.3 ml | acetonitrile | acetonitrile | chloroform | acetonitrile | 4/3/3 |
| 4-4 | 0.4 ml | — | 0.6 ml | acetonitrile | acetonitrile | — | acetonitrile | 4/0/6 |

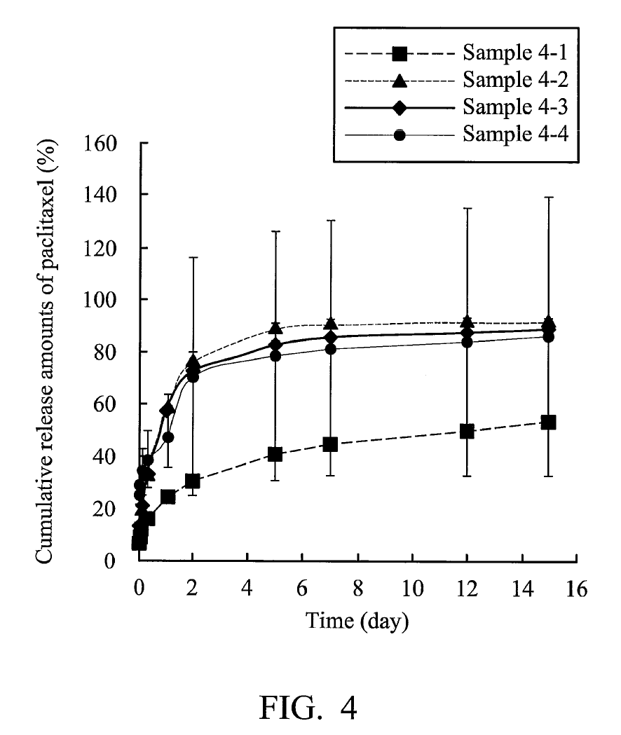

Subsequently, in vitro release tests were conducted. 10 mg of each of the above-mentioned samples was put into a vial filled with 20 ml of the modified PBS buffer (phosphate buffer saline, 4% CremoEL, and 2.4% Tween 80). At the time point for analyses, 20 ml of the modified PBS buffer in the vial was pipetted from the vial and the vial was replenished with another 20 ml of fresh modified PBS buffer. The pipetted sample solutions were tested with HPLC to analyze the concentrations of paclitaxel within them. The cumulative release amount of the active ingredient was expressed in terms of percentage, which was calculated as follows: the amount of paclitaxel in the buffer solution divided by that in the sample and multiplied by 100. The results are shown in FIG. 4.

According to the release profiles shown in FIG. 4, the composition comprising only PCL as the polymer substrate (Sample 4-1), that comprising a blend of PCL and PSA as the polymer substrate, that comprising a blend of PCL and PEG as the polymer substrate, and that comprising a blend of PAL, PEG, and PSA as the polymer substrate (Samples 4-2, 4-4, and 4-3, respectively) showed release profiles of the active ingredient (paclitaxel) with different initial burst releases at the beginning, followed by a slow releases with a release rate decreasing over time. According to FIG. 4, it is noted that different polymer substrate formulations can result in distinct change of the initial burst release rates, the release rates thereafter, and the according cumulative release amounts. For chemotherapeutic agents, the cumulative release amount of the chemotherapeutic substance must be brought to a certain level required for efficacious treatment within a short period of time, when implanted into the tumor or cancerous tissue. According to the present invention, by adjusting the polymer substrate formulation, the release amount of the chemotherapeutic substance can be controlled and brought to a level required for exhibiting therapeutic effect, within a short time period.

As shown in FIG. 4, Sample 4-1 released much less a cumulative amount of paclitaxel than Samples 4-2, 4-3, and 4-4 did. The release profiles overall showed a trend that, when PCL was blended with PSA and/or PEG, as the portion of PSA was more than the other(s), the initial burst release rate of the active ingredient (paclitaxel) and the according cumulative release amount increased (Sample 4-2>Sample 4-3>Sample 4-4).

### Example 5

### Preparation of the Composition Encapsulating Y₂O₃

PLGA, PCL, and PEG were respectively dissolved with the first solvents as listed in Table 5 to form polymer solutions with a concentration of 100 mg/ml. Y₂O₃ was dissolved with the second kind solvent as listed in Table 5 (acetonitrile) to form a Y₂O₃ solution with a concentration of 100 mg/ml. Mixed together the above-mentioned constituent polymer solutions according to the formulations (four formulations) as listed in Table 5 to form the polymer substrate solutions. For each formulation, 1 ml of the polymer blend solution was uniformly mixed with 0.1 ml of the above-mentioned Y₂O₃ solution. The mixture was then heated under 70° C. After the solvents were vaporized and the solid mixture was cooled, Samples 5-1˜5-4 as listed in Table 5 were obtained.

TABLE 5
The formulation of the polymer substrate use to encapsulate Y₂O₃

| | | | | Solvents | | | | PCL/PLGA/PEG |
|---|---|---|---|---|---|---|---|---|
| Sample | PCL | PLGA | PEG | doxorubicin | PCL | PLGA | PEG | (by weight) |
| 5-1 | 1.0 ml | — | — | acetonitrile | acetonitrile | — | — | 10/0/0 |
| 5-2 | 0.4 ml | 0.6 ml | — | acetonitrile | acetonitrile | chloroform | — | 4/6/0 |
| 5-3 | 0.4 ml | 0.3 ml | 0.3 ml | acetonitrile | acetonitrile | chloroform | acetonitrile | 4/3/3 |
| 5-4 | 0.4 ml | — | 0.6 ml | acetonitrile | acetonitrile | — | acetonitrile | 4/0/6 |

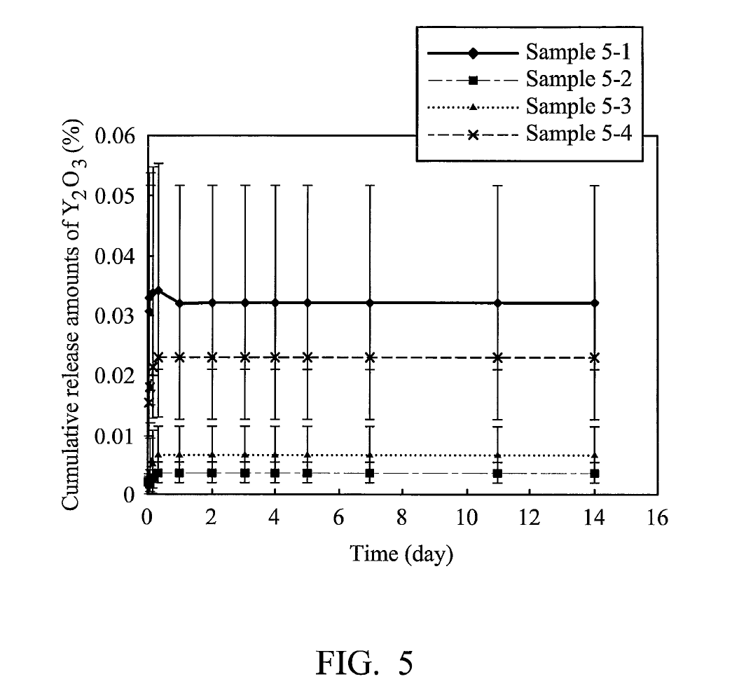

Subsequently, in vitro release tests were conducted. 10 mg of each of the above-mentioned samples was put into a vial filled with 20 ml of PBS buffer. At the time point for analyses, 20 ml of the PBS buffer in the vial was pipetted from the vial and the vial was replenished with another 20 ml of fresh PBS buffer. The pipetted sample solutions were tested with ICP-AES to analyze the concentrations of yttrium (Y) within them. The cumulative release amount of the active ingredient was expressed in terms of percentage, which was calculated as follows: the amount of yttrium (Y) in the buffer solution divided by that in the sample and multiplied by 100. The results are shown in FIG. 5. According to the release profiles shown in FIG. 5, the composition comprising only PCL as the polymer substrate (Sample 5-1), that comprising a blend of PCL and PSA as the polymer substrate, that comprising a blend of PCL and PEG as the polymer substrate, and that comprising a blend of PAL, PEG, and PSA as the polymer substrate (Samples 5-2, 5-4, and 5-3, respectively) showed release profiles of the active ingredient (Y₂O₃) with different initial burst release rates at the beginning and with slow release rates which were almost zero after the initial burst release. According to FIG. 5, it is noted that different polymer substrate formulations can result in distinct change of the initial burst release rates, the release rates thereafter, and the according cumulative release amount. For radiotherapeutic agents, the cumulative release amount of the radiotherapeutic substance for a specific period of time must be kept under a safety threshold level to prevent from damaging cells or tissues. According to the present invention, by modulating the formulation of the polymer substrate, the release amount of the radioactive agent can be controlled and kept under a safety threshold level and exhibit therapeutic effects.

As shown in FIG. 5, the release profiles of the active ingredient (Y₂O₃) showed a trend that the initial burst release rates and the according cumulative release amounts decreased as the ratio of the PCL portion in the polymer substrate decreased (Sample 5-2<Sample 5-3<Sample 5-4<Sample 5-1). It is also noted that, when the polymer substrate included a blend of PCL and PSA and/or PEG, the initial burst release rates and the according cumulative release amounts increased as the ratio of the PEG portion in the polymer substrate increased (Sample 5-2<Sample 5-3<Sample 5-4).

### Example 6

### Preparation of the Composition Encapsulating YCl₃

PLGA, PCL, and PEG were respectively dissolved with the first solvents as listed in Table 6 to form polymer solutions with a concentration of 100 mg/ml. YCl₃ was dissolved with the second kind solvent as listed in Table 6 (10% tetrahydrofuran (THF) aqueous solution, 90% by volume of THF in 10% by volume of water) to form a YCl₃ solution with a concentration of 100 mg/ml. Mixed together the above-mentioned constituent polymer solutions according to the formulations (four formulations) as listed in Table 6 to form the polymer substrate solutions. For each formulation, 1 ml of the polymer blend solution was uniformly mixed with 0.1 ml YCl₃ solution. The mixture was then heated under 70° C. After the solvents were vaporized and the solid mixture was cooled, Samples 6-1˜6-4 as listed in Table 6 were obtained.

TABLE 6
The formulation of the polymer substrate used to encapsulate YCl₃

| | | | | Solvents | | | | PCL/PLGA/PEG |
|---|---|---|---|---|---|---|---|---|
| Sample | PCL | PLGA | PEG | YCl₃ | PCL | PLGA | PEG | (by weight) |
| 6-1 | 1 ml | — | — | THF aq. | acetonitrile | — | — | 1/0/0 |
| 6-2 | 0.4 ml | 0.6 ml | — | THF aq. | acetonitrile | chloroform | — | 4/6/0 |
| 6-3 | 0.4 ml | 0.3 ml | 0.3 ml | THF aq. | acetonitrile | chloroform | acetonitrile | 4/3/3 |
| 6-4 | 0.4 ml | — | 0.6 ml | THF aq. | acetonitrile | — | acetonitrile | 4/0/6 |

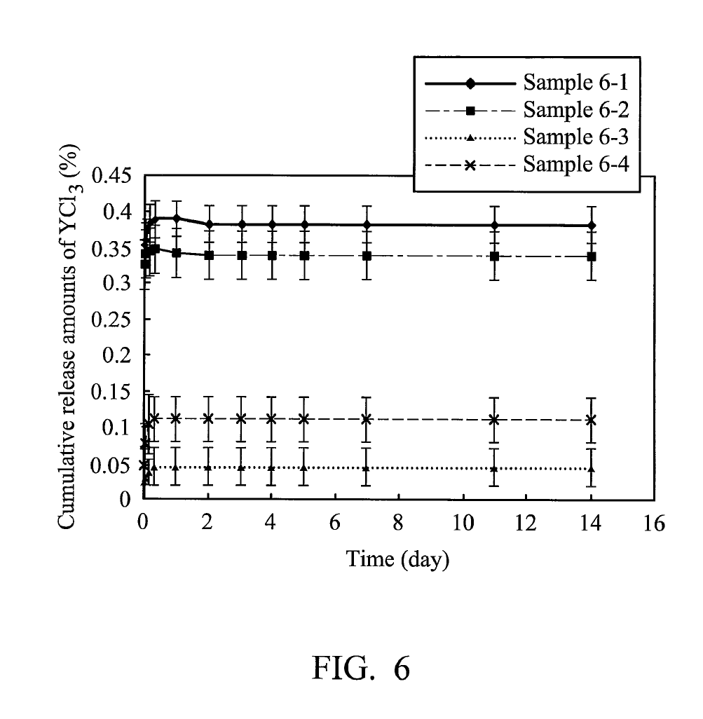

Subsequently, in vitro release tests were conducted. 10 mg of each of the above-mentioned samples was put into a vial filled with 20 ml of PBS buffer. At the time point for analyses, 20 ml of the PBS buffer in the vial was pipetted from the vial and the vial was replenished with another 20 ml of fresh PBS buffer. The pipetted sample solutions were tested with ICP-AES to analyze the concentrations of yttrium (Y) within them. The cumulative release amount of the active ingredient was expressed in terms of percentage, which was calculated as follows: the amount of yttrium (Y) in the buffer solution divided by that in the sample and multiplied by 100. The results are shown in FIG. 6. According to the release profiles shown in FIG. 6, the composition comprising only PCL as the polymer substrate (Sample 6-1) and that comprising a blend of PCL and PSA as the polymer substrate, that comprising a blend of PCL and PEG as the polymer substrate, and that comprising a blend of PAL, PEG, and PSA as the polymer substrate (Samples 6-2, 6-4, and 6-3, respectively) showed release profiles of the active ingredient (YCl₃) with different initial burst release rates at the beginning and with slow release rates which were almost zero after the initial burst release. According to FIG. 6, it is noted that different polymer substrate formulations can result in distinct change of the initial burst release rates, the release rates thereafter, and the according cumulative release amount. For radiotherapeutic agents, the cumulative release amount of the radiotherapeutic substance for a specific period of time must be kept under a safety threshold level to prevent from damaging cells or tissues. According to the present invention, by modulating the formulation of the polymer substrate, the release amount of the radioactive agent can be controlled and kept under a safety threshold level and exhibit therapeutic effects.

As shown in FIG. 6, the release profiles of the active ingredient (YCl₃) showed a trend that, the initial burst release rate and the according cumulative release amount increased as the ratio of the PCL portion in the polymer substrate increased (Sample 6-1). It is also noted that, when the polymer substrate included a blend of PCL and PSA and/or PEG, the initial burst release rates and the cumulative release amounts decreased as the ratio of the PEG portion in the polymer substrate increased (Sample 6-4, compared to Sample 6-2). When the polymer blend included the same amounts of PEG and PSA, the initial burst release rate was the slowest and the according cumulative release amount was the lowest (Sample 6-3, compared to Samples 6-2 and 6-4).

### Example 7

### In Vitro Disintegration Test

10 mg of each samples (Samples 1-1, 2-1, 2-3, 2-4, 2-5, 2-8, 2-10, and the control sample consisted of only PCL (Sample 1-5)) were respectively put into a vial filled 20 ml of phosphate buffer. At the time point for analyses, removed the phosphate buffers, placed the samples in a vacuum dryer for 24 hours, and weighted the samples after they were dried. The weight loss was expressed in terms of percentage, which was calculated as with the following formula.

Weight loss (%)=(W₀−Wₜ)/W₀×100%

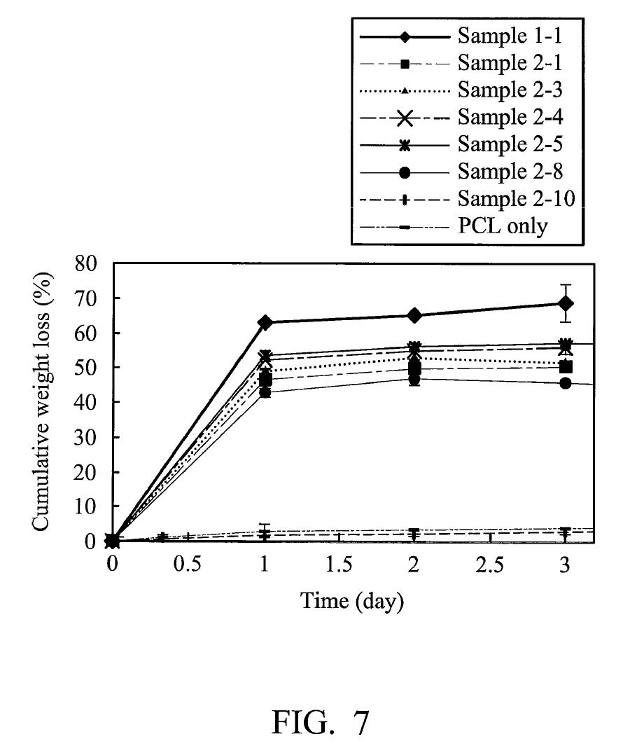

W₀ represents the dry weight of the sample before being put into the phosphate buffer. Wₜ represents the dry weight of the sample taken out from the phosphate buffer. The results are shown in FIG. 7.

As shown in the weight loss profiles shown in FIG. 7, the control sample consisted of only PCL (Sample 1-5) and Sample 1-1 consisted of PCL and encapsulated NaReO₄ were hardly disintegrated. While, the samples comprising polymer blend and encapsulated active ingredient, like Sample 2-1 (with a blend of about 50 wt % of PCL and about 50 wt % of PSA as the polymer substrate and Re—Sn as the active ingredient), Sample 2-3 (with a blend of about 40 wt % of PCL and about 60 wt % of PSA as the polymer substrate and Re—Sn as the active ingredient), Sample 2-4 (with a blend of about 40 wt % of PCL, about 10 wt % of PSA and about 50 wt % of PEG as the polymer substrate and Re—Sn as the active ingredient), Sample 2-5 (with a blend of about 40 wt % of PCL, about 40 wt % of PSA, and about 20 wt % of PEG as the polymer substrate and Re—Sn as the active ingredient), Sample 2-8 (with a blend of about 40 wt % of PCL, about 50 wt % of PSA, and about 10 wt % of PEG as the polymer substrate and Re—Sn as the active ingredient), and Sample 2-10 (with a blend of about 30 wt % of PCL, about 40 wt % of PSA, and about 30 wt % of PEG as the polymer substrate and Re—Sn as the active ingredient), were disintegrated by 40%˜60% by weight on the first day.

According to the present invention, the polymer substrate formulation may be appropriately modulated for optimum therapeutic effects, based on the chemical and physical properties of the encapsulated active ingredient, the treatment design, or other factors.

While the invention has been described by way of examples and in terms of the preferred embodiments, it is to be understood that the present invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A controllable release composition, comprising a mixture of a polymer substrate and a radioactive agent, wherein the polymer substrate is a polymer blend comprising a biodegradable polyester and polyanhydride.

2. The composition as claimed in claim 1, wherein the polyester comprises polycaprolactone (PCL), polyvalerolactone (PVL), polypropiolactone (PPL), polybutyrolactone (PBL), poly(lactide-co-glycolide (PLGA), polylactic acid (PLA), polyglycolide (PGA), poly(isobutylcyanoacrylate (PIBCA), polyisophthalic acid (PIPA), poly-1,4-phenylene dipropionic acid (PPDA), poly(mandelic acid) (PMDA), poly(propylene fumarate (PPF), poly(ortho ester) (POE), or combinations thereof.

3. The composition as claimed in claim 1, wherein the polyanhydride comprises poly(sebacic anhydride) (PSA), poly-(bis-(p-carboxyphenoxy)propane anhydride (PCPPA), poly-(bis(p-carboxy)methane anhydride) (PCMA), poly-carboxyphenoxypropane-co-sebasic acid (p(CPP-SA)), poly-carboxyphenoxypropane-co-isophthalic acid (p(CPP-IPA)), poly(fatty acid dimmer-co-sebacic acid (p(FAD-SA)), or combinations thereof.

4. The composition as claimed in claim 1, wherein the polymer blend comprises 50 parts by weight of the polyester and 5˜70 parts by weight of the polyanhydride.

5. The composition as claimed in claim 1, wherein the polymer blend further comprises a biodegradable polyether.

6. The composition as claimed in claim 5, wherein the polyether comprises poly(ethylene glycol) (PEG), poly(propylene glycol) (PPG), poly(butylenes glycol) (PBG), or combinations thereof.

7. The composition as claimed in claim 5, wherein the polymer blend comprises 50 parts by weight of the polyester, 5˜70 parts by weight of the polyanhydride, and 5˜70 parts by weight of the polyether.

8. The composition as claimed in claim 1, wherein the radioactive agent comprises a radioactive seed of Co-60, Sr-89, I-125, Cs-137, Ir-192, Y-90, Re-188, or Ra-226.

9. The composition as claimed in claim 1, used for solid tumor treatment.

10. The composition as claimed in claim 9, wherein the solid tumor treatment comprises brachytherapy and/or implantation therapy.

11. The composition as claimed in claim 1, wherein the composition is in an implant dosage form.

12. The composition as claimed in claim 1, wherein the polymer blend and the radioactive agent are present in a ratio of 100:0.0002˜100:22 by weight.

13. A controllable release composition, comprising a mixture of a polymer substrate and a chemotherapeutic agent, wherein the polymer substrate is a polymer blend comprising a biodegradable polyester, polyanhydride, and polyether.

14. The composition as claimed in claim 13, wherein the polyester comprises polycaprolactone (PCL), polyvalerolactone (PVL), polypropiolactone (PPL), polybutyrolactone (PBL), poly(lactide-co-glycolide (PLGA), polylactic acid (PLA), polyglycolide (PGA), poly(isobutylcyanoacrylate (PIBCA), polyisophthalic acid (PIPA), poly-1,4-phenylene dipropionic acid (PPDA), poly(mandelic acid) (PMDA), poly(propylene fumarate (PPF), poly(ortho ester) (POE), or combinations thereof.

15. The composition as claimed in claim 13, wherein the polyanhydride comprises poly(sebacic anhydride) (PSA), poly-(bis-(p-carboxyphenoxy)propane anhydride (PCPPA), poly-(bis(p-carboxy)methane anhydride) (PCMA), poly-carboxyphenoxypropane-co-sebasic acid (p(CPP-SA)), poly-carboxyphenoxypropane-co-isophthalic acid (p(CPP-IPA)), poly(fatty acid dimmer-co-sebacic acid (p(FAD-SA)), or combinations thereof.

16. The composition as claimed in claim 13, wherein the polyether comprises poly(ethylene glycol) (PEG), poly(propylene glycol) (PPG), poly(butylenes glycol) (PBG), or combinations thereof.

17. The composition as claimed in claim 13, wherein the polymer blend comprises 50 parts by weight of the polyester, 5˜70 parts by weight of the polyanhydride, and 5˜70 parts by weight of the polyether.

18. The composition as claimed in claim 13, wherein the chemotherapeutic agent comprises 5-fluorouracil, irinotecan, topotecan, bortezomib, lapatinib, trastuzumab, gemcitabine, methotrexate, doxorubicin, oxaliplatin, paclitaxel, camptothecin, cisplatin, bevacizumab, or combinations thereof.

19. The composition as claimed in claim 13, used for solid tumor treatment.

20. The composition as claimed in claim 19, wherein the solid tumor treatment comprises implantation therapy and/or brachytherapy.

21. The composition as claimed in claim 13, wherein the composition is in an implant dosage form.

22. The composition as claimed in claim 13, wherein the polymer blend and the chemotherapeutic agent are present in a ratio of 100:0.0002˜400:22 by weight.

23. A method for preparing the controllable release composition as claimed in claim 1, comprising:
dissolving or dispersing the polymer blend with a first solvent to form a first solution or suspension,
dissolving or dispersing the radioactive agent with a second solvent to form a second solution or suspension,
mixing the first solution or suspension and the second solution or suspension to form a mixture,
heating the mixture to vaporize the first and second solvent, and
obtaining the controllable release composition.

24. The method as claimed in claim 23, wherein the first solvent comprises acetone, acetonitrile, chloroform, dichloromethane, ethyl acetate, isopropanol, methanol, tetrahydrofuran, or combinations thereof.

25. The method as claimed in claim 23, wherein the second solvent comprises water, acetone, acetonitrile, chloroform, dichloromethane, ethyl acetate, isopropanol, methanol, ethanol, tetrahydrofuran, or combinations thereof.

26. The method as claimed in claim 23, wherein the polyester comprises polycaprolactone (PCL), polyvalerolactone (PVL), polypropiolactone (PPL), polybutyrolactone (PBL), poly(lactide-co-glycolide (PLGA), polylactic acid (PLA), polyglycolide (PGA), poly(isobutylcyanoacrylate (PIBCA), polyisophthalic acid (PIPA), poly-1,4-phenylene dipropionic acid (PPDA), poly(mandelic acid) (PMDA), poly(propylene fumarate (PPF), poly(ortho ester) (POE), or combinations thereof.

27. The method as claimed in claim 23, wherein the polyanhydride comprises poly(sebacic anhydride) (PSA), poly-(bis-(p-carboxyphenoxy)propane anhydride (PCPPA), poly-(bis(p-carboxy)methane anhydride) (PCMA), poly-carboxyphenoxypropane-co-sebasic acid (p(CPP-SA)), poly-carboxyphenoxypropane-co-isophthalic acid (p(CPP-IPA)), poly(fatty acid dimmer-co-sebacic acid (p(FAD-SA)), or combinations thereof.

28. The method as claimed in claim 23, wherein the polymer blend comprises 50 parts by weight of the polyester and 5-70 parts by weight of the polyanhydride.

29. The method as claimed in claim 23, wherein the polymer blend further comprises a biodegradable polyether.

30. The method as claimed in claim 29, wherein the polyether comprises poly(ethylene glycol) (PEG), poly(propylene glycol) (PPG), poly(butylenes glycol) (PBG), or combinations thereof.

31. The method as claimed in claim 29, wherein the polymer blend comprises 50 parts by weight of the polyester, 5˜70 parts by weight of the polyanhydride, and 5˜70 parts by weight of the polyether.

32. The method as claimed in claim 23, wherein the radioactive agent comprises a radioactive seed of Co-60, Sr-89, I-125, Cs-137, Ir-192, Y-90, Re-188 or Ra-226.

33. The method as claimed in claim 23, wherein the polymer blend and the radioactive agent are present in a ratio of 100:0.0002˜400:22 by weight.

34. The method as claimed in claim 23, wherein the heating is at a temperature of 40˜80° C.

35. A method for preparing the controllable release composition as claimed in claim 13, comprising:
dissolving or dispersing the polymer blend with a first solvent to form a first solution or suspension,
dissolving or dispersing the chemotherapeutic agent with a second solvent to form a second solution or suspension,
mixing the first solution or suspension and the second solution or suspension to form a mixture,
heating the mixture to vaporize the first and second solvent, and
obtaining the controllable release composition.

36. The method as claimed in claim 35, wherein the first solvent comprises acetone, acetonitrile, chloroform, dichloromethane, ethyl acetate, isopropanol, methanol, tetrahydrofuran, or combinations thereof.

37. The method as claimed in claim 35, wherein the second solvent comprises water, acetone, acetonitrile, chloroform, dichloromethane, ethyl acetate, isopropanol, methanol, ethanol, tetrahydrofuran, or combinations thereof.

38. The method as claimed in claim 35, wherein the polyester comprises polycaprolactone (PCL), polyvalerolactone (PVL), polypropiolactone (PPL), polybutyrolactone (PBL), poly(lactide-co-glycolide (PLGA), polylactic acid (PLA), polyglycolide (PGA), poly(isobutylcyanoacrylate (PIBCA), polyisophthalic acid (PIPA), poly-1,4-phenylene dipropionic acid (PPDA), poly(mandelic acid) (PMDA), poly(propylene fumarate (PPF), poly(ortho ester) (POE), or combinations thereof.

39. The method as claimed in claim 35, wherein the polyanhydride comprises poly(sebacic anhydride) (PSA), poly-(bis-(p-carboxyphenoxy)propane anhydride (PCPPA), poly-(bis(p-carboxy)methane anhydride) (PCMA), poly-carboxyphenoxypropane-co-sebasic acid (p(CPP-SA)), poly-carboxyphenoxypropane-co-isophthalic acid (p(CPP-IPA)), poly(fatty acid dimmer-co-sebacic acid (p(FAD-SA)), or combinations thereof.

40. The method as claimed in claim 35, wherein the polyether comprises polyethylene glycol) (PEG), poly(propylene glycol) (PPG), poly(butylenes glycol) (PBG), or combinations thereof.

41. The method as claimed in claim 35, wherein the polymer blend comprises 50 parts by weight of the polyester, 5˜70 parts by weight of the polyanhydride, and 5˜70 parts by weight of the polyether.

42. The method as claimed in claim 35, wherein the chemotherapeutic agent comprises 5-fluorouracil, irinotecan, topotecan, bortezomib, lapatinib, trastuzumab, gemcitabine, methotrexate, doxorubicin, oxaliplatin, paclitaxel, camptothecin, cisplatin, bevacizumab, or combinations thereof.

43. The method as claimed in claim 35, wherein the polymer blend and the chemotherapeutic agent are present in a ratio of 100:0.0002˜100:22 by weight.

44. The method as claimed in claim 35, wherein the heating is at a temperature of 40˜80° C.
